(19) United States
(12) Patent Application Publication
(10) Pub. No.: US 2026/0069231 A1
(43) Pub. Date: Mar. 12, 2026

(54) DETERMINATION OF RESPIRATORY WAVEFORM FROM SINGLES RATES

(71) Applicant: Siemens Medical Solutions USA, Inc., Malvern, PA (US)

(72) Inventor: James Hamill, Knoxville, TN (US)

(21) Appl. No.: 19/303,440

(22) Filed: Aug. 19, 2025

Related U.S. Application Data
(60) Provisional application No. 63/693,233, filed on Sep. 11, 2024.

(51) Int. Cl. A61B 6/00; A61B 6/03; G01T 1/29; G06T 11/00

(52) U.S. Cl. CPC A61B 6/5264; A61B 6/037; G01T 1/2985; G06T 12/10; A61B 6/5205; G06T 2210/41

(57) ABSTRACT

A system and method comprise acquisition of data representing singles received by regions of detector crystals, determination of time-series data of a count of singles detected by the detector crystals of each region, determination of a representative region based on the time-series data of each region, determination of a correlation between the time-series data of the representative region and time-series data of the count of singles of each other region, generation of a motion signal based on the time-series data of the representative region and the time-series data of the other regions based on the determined correlations, determination of coincidences corresponding to selected periods of the motion signal, and reconstruction of an image based on the determined coincidences.

### CROSS-REFERENCE TO RELATED APPLICATIONS

The present application claims priority to, and the benefit of, U.S. Provisional Patent Application No. 63/693,233, filed Sep. 11, 2024, the contents of which are incorporated by reference herein for all purposes.

### BACKGROUND

Nuclear medicine uses radiation emission to acquire images that illustrate the function and physiology of organs, bones or tissues of the body. According to positron-emission-tomography (PET) imaging, a radiopharmaceutical tracer is introduced into a patient body via arterial injection. Radioactive decay of the tracer generates positrons which eventually encounter electrons and are annihilated thereby. The annihilation produces two 511 keV photons which travel in approximately opposite directions.

A ring of detectors surrounding the body detects photons, identifies “coincidences” based thereon, and reconstructs PET images based on the identified coincidences. A coincidence is identified when two photons (i.e., two “singles” or, alternatively, “events”) arrive at two detector crystals disposed on opposite sides of the body within a short time window, indicating that the two photons arose from the same positron annihilation. Because the two photons travel in approximately opposite directions, the locations of the two detector crystals determine a line-of-response (LOR) along which an annihilation may have occurred. Time-of-flight (TOF) PET additionally measures the difference between the arrival times of the two photons arising from the annihilation. This difference may be used to estimate a particular position along the LOR at which the annihilation occurred.

The quality of a reconstructed PET image may be degraded due to respiratory motion during photon detection. The motion may result in a blurred torso region and limit detectability of small or low-contrast lesions. Respiratory gating attempts to reduce image degradation due to respiratory motion by separating the breathing cycle into different phases and generating phase-specific images from PET data which was acquired during the different phases. Respiratory gating requires a time-series waveform of respiratory motion in order to correlate acquired PET data with different respiratory phases based on the time at which the PET data was acquired.

A time-series waveform of respiratory motion (i.e., a respiratory signal) is typically acquired during PET data acquisition via external devices (e.g., cameras, pressure belts, strain gauges). These hardware-based methods require additional setup time and effort, and the resulting image quality can vary greatly depending on the quality of the setup. External devices may also add to the discomfort already experienced by a patient within the PET scanner.

Known “data-driven” systems attempt to generate a signal representing respiratory motion based on the acquired PET data. Some of these systems rely on an analysis of a reconstructed time-series of PET images. For example, these systems identify coincidences which occurred during each of many time periods, reconstruct an image for each time period based on the coincidences which occurred during the time period, and determine a respiratory signal based on the voxels of each reconstructed image. These coincidence-based methods are time- and resource-consuming, as they require analysis of millions of singles and sorting of the coincidences into fine-scale four dimensional sinograms. In another example, some systems attempt to generate a signal representing respiratory motion based on detected singles. However, the signals generated by these latter systems are often noisy, inaccurate, and/or otherwise unsuitable for use in respiratory gating.

Systems are desired to efficiently and accurately estimate respiratory motion based on acquired PET data.

### BRIEF DESCRIPTION OF THE DRAWINGS

The foregoing and other aspects of some embodiments are best understood from the following detailed description when read in connection with the accompanying drawings. Embodiments are not limited to the specific implementations disclosed herein.

FIGS. 1A and 1B illustrate the interaction of photons with a PET detector ring of a PET scanner according to some embodiments;

FIG. 2 illustrates a PET detector ring and image reconstruction components of a PET scanner according to some embodiments;

FIG. 3 is a flow diagram of a process to generate a signal representing respiratory motion from singles data according to some embodiments;

FIG. 4 illustrates detector crystals of a PET detector ring according to some embodiments;

FIG. 5 illustrates regions of detector crystals of a PET detector ring according to some embodiments;

FIG. 6 illustrates an average count of singles per unit time for each region of detector crystals according to some embodiments;

FIG. 7 illustrates a variation of the singles count time-series data for each region of detector crystals according to some embodiments;

FIG. 8 illustrates correlations between region singles count time-series data associated with a largest variation and the singles count time-series data for each region of detector crystals according to some embodiments;

FIG. 9 illustrates a signal representing respiratory motion generated based on correlation-weighted singles count time-series data of each detector region according to some embodiments;

FIG. 10 is a flow diagram of a process to generate a PET image according to some embodiments; and

FIG. 11 is a block diagram of a PET-CT imaging system according to some embodiments.

### DETAILED DESCRIPTION

The following description is provided to enable any person in the art to make and use the described embodiments. Various modifications, however, will be readily-apparent to those in the art.

Some embodiments provide efficient and accurate estimates of respiratory motion based on detected singles. Briefly, time-series data of a count of singles detected by each of a plurality of PET detector crystals is determined, a motion-representative detector region is determined based on the time-series data, correlations are determined between the time-series data determined for the motion-representative detector region and time-series data determined for each of a plurality of other detector regions, and the time-series data determined for the motion-representative detector region and the time-series data determined for each of the other detector regions are combined based on the determined correlations. The combined time-series data is a signal representing respiratory motion which occurred during acquisition of the time-series data.

Accordingly, time periods which correspond to a phase of respiratory motion (e.g., end of exhalation) may be determined based on the amplitude of the combined time-series data, coincidences associated with each time period may be identified (i.e., based on their timestamps), and a motion-corrected image may be reconstructed based on the coincidences.

The correlations may indicate a positive or negative correlation between the time-series data of the motion-representative detector region and the time-series data of another detector region. A positive correlation may indicate that a detector region is located on a same side of the PET scanner as the motion-representative detector region with respect to the patient's respiratory motion. Accordingly, when the patient's body moves closer to the motion-representative detector region, it also moves closer to detector regions whose time-series data are positively correlated with the time-series of the motion-representative detector region. In contrast, when the patient's body moves closer to the motion-representative detector region, it moves away from detector regions whose time-series data are negatively correlated with the time-series data of the motion-representative detector region.

The combination of the time-series data based on the correlations may result in the positively-correlated time-series data being combined directly (e.g., in-phase) and the negatively-correlated time-series data being inverted such that the positively-correlated time-series data is combined with the inverse of each of the negatively-correlated time-series data. Advantageously, this correlation-driven combination results in a signal exhibiting a higher signal-to-noise ratio and greater accuracy with respect to actual respiratory motion than other data-driven systems.

PET detectors typically detect singles over a larger spectrum of energy ranges. Since PET imaging relies on the detection of 511 keV photons, singles of other energy ranges are ignored during the determination of coincidences. However, the present inventors have recognized that low energy singles (e.g., 150-250 keV) can be 10 to 1000 times more plentiful than ˜511 keV singles during a typical PET scan (e.g., when using an yttrium-90 tracer) and, rather than being ignored, can be used in the processes described herein to increase the signal-to-noise ratio of the resulting respiratory waveform.

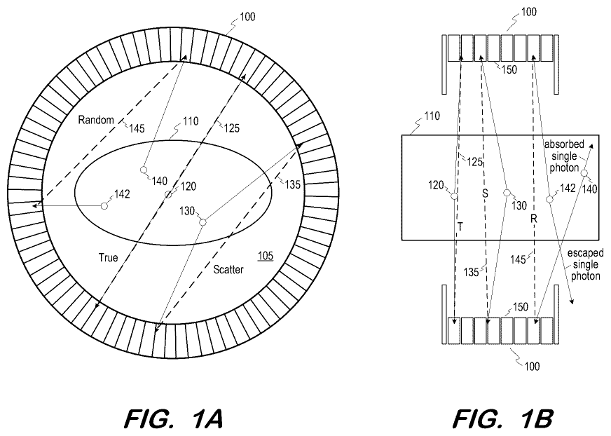

FIG. 1A and FIG. 1B illustrate detection of singles and coincidences by a PET scanner according to some embodiments. FIG. 1A is a transaxial view of bore 105 of PET scanner detector ring 100 and imaging subject 110 disposed therein. Imaging subject 110 may comprise a human body, a phantom, or any other suitable subject. FIG. 1B is an axial view of detector ring 100 and subject 110 of FIG. 1A. Detector ring 100 is composed of an arbitrary number (eight in this example) of adjacent and coaxial rings of detectors 150 in the illustrated example. Each detector 150 may comprise any number of detector crystals and electrical transducers.

The detector crystals may comprise lutetium oxyorthosilicate (LSO), lutetium-yttrium oxyorthosilicate (LYSO), or any other suitable materials that are or become known. According to some embodiments, the electrical transducers may comprise silicon photomultipliers (SiPMs) or photomultiplier tubes (PMTs). The detector crystals create light photons in response to receiving gamma photons. The electrical transducers, or photosensors, convert these light photons to electrical pulses.

In some implementations, each detector crystal is optically coupled to one specific SiPM transducer so all pulses generated by the transducer are assumed to have been caused by a photon received at its corresponding detector crystal. According to other implementations, more than one electrical transducer may receive light generated by a detector crystal. A weighted sum of the corresponding electrical pulses may be used to estimate an interaction position, and the crystal closest to the estimated position is determined to correspond to the received photon. According to light-sharing techniques, in which scintillation light is also spread over multiple detectors, a pre-computed look-up table maps pulse patterns to specific detector crystals.

As an alternative to the above-described scintillator-based detectors, a direct conversion PET detector uses a semiconductor such as CdZnTe or HgI₂ to generate electrical pulses without the use of a scintillator. The semiconductor absorbs an incoming gamma photon, generates electron-hole pairs, and drifts the charges to electrodes under an applied bias, from which the induced current is read as an electrical pulse proportional to the energy of the gamma photon.

Regardless of whether an electrical pulse is generated by scintillation or direct conversion, the electrical pulse is processed to ensure it represents a gamma energy within a desired range and an event time is derived therefrom. The event time represents the time at which its corresponding photon was “detected”. Each single is captured as PET singles data indicating a detector crystal at which a photon was received, an event time, and possibly other data (e.g., pulse energy, pulse amplitude).

Annihilations 120, 130, 140 and 142 are assumed to occur at various locations within subject 110. As described above, an injected tracer generates positrons which are annihilated by electrons to produce two 511 keV photons which travel in approximately opposite directions. Each of annihilations 120, 130, 140 and 142 results in the detection of a coincidence. True coincidences represent valid image data, while scatter and random coincidences represent noise associated with incorrect event position information.

Coincidences are detected based on PET singles data, also known as PET event data. A coincidence is detected when the event times of two ˜511 keV singles are within a specified coincidence time window of one another. For example, annihilation 120 resulted in two photons which were detected within the coincidence time window. These detections represent a true coincidence because the position of annihilation 120 lies on LOR 125 which connects the positions of the detector crystals at which the two photons were received.

Annihilation 130 results in scatter coincidence because, even though the two photons resulting from annihilation 130 were detected within the coincidence time window, the position of annihilation 130 does not lie on LOR 135 connecting the two crystals which received the photons. This may be due to Compton (i.e., inelastic) or Coherent (i.e., elastic) scatter resulting in a change of direction of at least one of the two photons within subject 110.

Annihilations 140 and 142 are two separate annihilations which result in detection of a random coincidence. In the present example, one of the photons generated by annihilation 140 is absorbed in body 210 and one of the photons generated by annihilation 142 escapes detection by any detector 150 of detector ring 100. The remaining two photons generated by the two annihilations happen to be detected within the coincidence time window, even though no annihilation occurred on LOR 145 connecting the positions at which the coincident photons were received.

The detected coincidences may be stored as PET coincidence data comprising raw (i.e., list-mode) data and/or sinograms. List-mode data may represent each coincidence via data identifying the two detector crystals which define the LOR of the coincidence and the event times of the electrical pulses of the coincidence. Since only the true unscattered coincidences indicate locations of actual annihilations, random coincidences and scatter coincidences are often subtracted from or otherwise used to correct the PET coincidence data prior to or during reconstruction of a PET image based thereon.

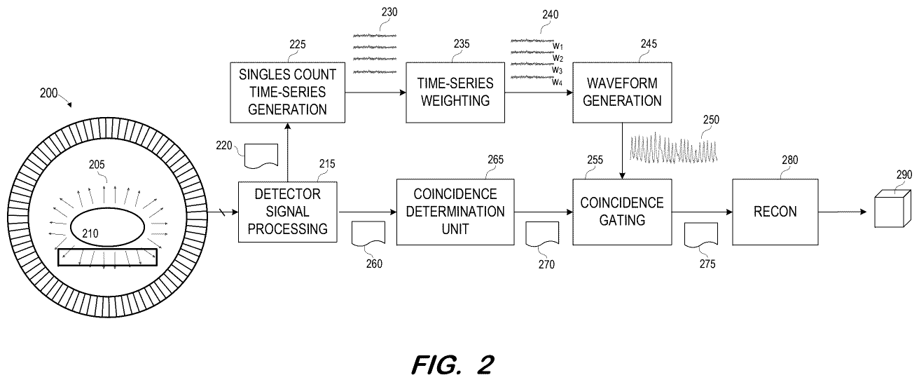

FIG. 2 illustrates PET detector ring 200 of a PET scanner according to some embodiments. Detector ring 200 includes a plurality of detectors in the axial direction as well as the illustrated detectors in the transaxial direction. Detector ring 200 receives photons 205 emitted from volume 210. As described above, the detectors of detector ring 200 generate electrical signals based on the energy of the received photons.

Detector signal processing unit 215 is configured to receive electrical pulses from detector ring 200, to reject pulses outside a designated energy window (i.e., invalid pulses), and generate PET singles data 220 from the remaining electrical pulses. According to some embodiments, the designated energy window includes lower energies than a typical energy window used during a PET scan. For example, the designated energy window may include energies from 150 keV to 650 keV, in order to acquire significantly more singles data than a typical energy window of 425 keV-650 keV. PET singles data 220 may comprise list-mode data indicating a detector crystal, an event time, an energy, etc. for each valid received photon. Embodiments may utilize any PET scanner which provides PET singles data via list-mode data, sinograms, or other formats.

PET singles data 220 may be acquired in parallel with PET singles data which will be used to identify coincidences. Some PET scanners include components, known as scalers, which output the singles rate per detector block. Since, unlike the above-described singles data, the singles data output by a scaler is not used to identify coincidences, the singles data output by a scaler need not identify a specific crystal (or sub-crystal position). Rather, the singles data output by a scaler may include an identifier of a detector block at which a photon was received, an event time, and other data (e.g., pulse energy, pulse amplitude). Some embodiments use the detector block-specific singles data acquired from scalers, and not crystal-specific singles data used to identify coincidences, to generate a motion signal as described herein.

Singles count time-series generation component 225 receives PET singles data 220 and generates time-series data therefrom. Each component depicted in FIG. 2 may be implemented using any combination of hardware and/or software (i.e., executable program code). Two or more components of FIG. 2 may be implemented using the same hardware and/or software.

According to some embodiments, singles count time-series generation component 225 determines, based on PET singles data 220 and for each detector crystal of ring 200, a count of singles detected by the detector crystal during each of a series of consecutive time periods. For example, the time-series data for a particular detector crystal may indicate a count of 5,000 over a first 100 ms, a count of 4500 over a next 100 ms, and a count of 5500 over a next 100 ms. The time-series data therefore depicts a singles rate rather than a cumulative count of singles.

Singles count time-series generation component 225 may smooth the time-series data according to any suitable smoothing algorithm. Singles count time-series generation component 225 may subtract from each set of time-series data an average value of a portion of the time-series data. Such smoothing and offset removal may facilitate the below-described subsequent processing of the time-series data.

Singles count time-series generation component 225 may also determine singles count time-series data for each detector region of detector ring 200. Each detector crystal may be assigned to a detector region such that, for example, each detector region consists of an area of 40×40 detector crystals. The singles count time-series data for a detector region is equal to a sum of the singles count time-series data for each detector crystal in the detector region. Singles count time-series generation component 225 may determine singles count time-series data for each detector region based on detector block-specific singles data acquired from scalers. A detector region may consist of eight contiguous detector blocks each consisting of 10×20 detector crystals. In this case, the singles count time-series data for a detector region is equal to a sum of the singles count time-series data for each detector block in the detector region. Component 225 outputs the region singles count time-series data 230 for each detector region to time-series weighting component 235.

Time-series weighting component 235 determines correlations between detector regions based on the singles count time-series data 230 for each detector region. For example, time-series weighting component 235 may a determine detector region which most-strongly indicates movement based on the singles count time-series data 230, and determine correlations between the singles count time-series data of each detector region and the singles count time-series data of the determined detector region. A correlation may indicate whether singles count time-series data of a region tends to move in a same direction as (i.e., is positively correlated with) or in an opposite direction as (i.e., is negatively correlated with) the singles count time-series data of the determined detector region.

Time-series weighting component 235 weights each singles count time-series data 230 based on the correlations. According to some embodiments, singles count time-series data 230 of a region which is positively correlated with the singles count time-series data of the determined detector region is assigned a positive weighting and singles count time-series data 230 of a region which is negatively correlated with the singles count time-series data of the determined detector region is assigned a negative weighting. The magnitudes of the assigned weightings may depend on the degree of (positive or negative) correlation.

Waveform generation component 245 combines weighted time-series data 240 according to the weightings. In one example, waveform generation component 245 multiples each of time-series data 230 by its weighting and sums all resulting products. Waveform generation component 245 may change a sign of the summed signal if needed to ensure that the signal peaks represent inhale periods and the signal valleys represent exhale periods. A baseline adjustment may be applied such that all (or most) values of the summed signal are positive values. Resulting waveform 250 is output to coincidence gating unit 255 to perform gating as is known in the art.

In this regard, coincidence determination unit 265 identifies a coincidence event for each pair of singles within PET singles data 260 whose event times fall within a coincidence time window. Detector signal processing unit 215 may be configured to generate PET singles data 260 based only on singles falling within an energy window of 425 keV-650 keV. In other embodiments, coincidence determination unit 265 receives detector crystal-specific PET singles data 220 as described above and ignores low-energy singles during coincidence determination. As mentioned above, PET singles data 260 may be acquired independently from acquisition of detector block-specific singles data 220. Coincidence determination unit 265 outputs PET coincidence data 270, which may comprise list-mode data, sinogram data, etc.

Coincidence gating component 255 uses waveform 250 to determine time periods during the data acquisition which correspond to a desired phase of respiratory motion. In some examples, coincidence gating component 255 identifies periods during which an amplitude of signal 250 (and therefore patient motion) is lowest. These periods may correspond to an end of an exhalation phase of the respiratory cycle. Coincidence gating component 255 identifies coincidence data 275 of coincidence data 270 having timestamps falling within the identified periods.

Reconstruction component 280 applies any suitable reconstruction algorithm that is or becomes known to coincidence data 275 to generate image volume 290. The reconstruction algorithm may comprise filtered backprojection (FBP) or ordered subsets expectation maximization (OSEM), but embodiments are not limited thereto. Reconstruction may include any other suitable steps, such as subtraction of random coincidences and scatter coincidences, motion correction, attenuation correction using a linear attenuation coefficient map, and correction for system sensitivity.

The desired-phase (i.e., low motion) image 290 described above typically represents approximately one-third of the determined coincidences. To increase the signal-to-noise ratio, further embodiments may reconstruct an image for each other phase of motion from coincidences occurring during those phases. Each of these images is mapped to the desired-phase (i.e., low motion) image (e.g., using an optical flow algorithm) to determine how each voxel moves during the breathing cycle. The images are spatially warped based on their mappings and all the warped images are summed with the desired-phase image to produce a single image representing all the determined coincidences.

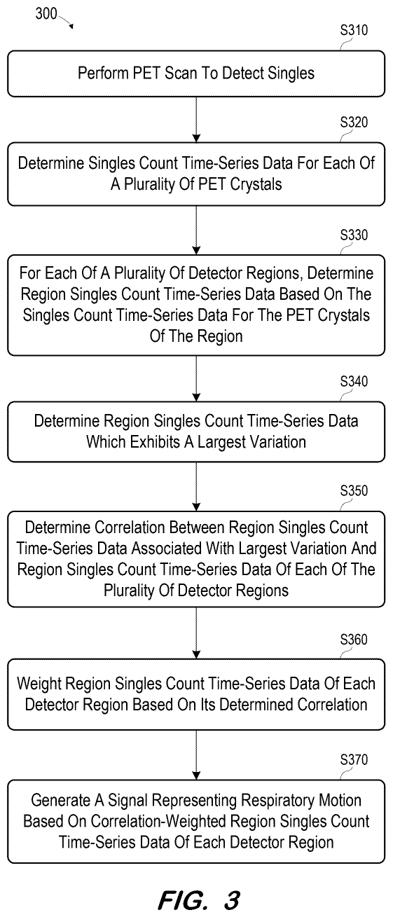

FIG. 3 is a flow diagram of process 300 to determine a signal representing respiratory motion from PET singles data according to some embodiments. Process 300 may be performed by any combination of hardware and software that is or becomes known. Program code embodying these processes may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processor cores, and processor threads. Embodiments are not limited to the examples described below.

Initially, a PET scan is performed at S310 to detect singles and generate corresponding PET singles data. A radiopharmaceutical tracer (e.g., yttrium-90) is introduced into a patient body via arterial injection. Detector crystals surrounding the patient receive photons due to decay of the tracer and directly emit electrical signals or emit light photons which are converted to electrical signals by adjacent photosensors. The electrical signals are processed to generate PET singles data which indicates, for each detected single, a detector crystal which received the photon, an event time, and other needed information. As described above, some embodiments include PET singles data processed from electrical signals resulting from low energy (e.g., 150 keV to 425 keV) photons and from higher-energy (e.g., 425 keV to 1000 keV) photons.

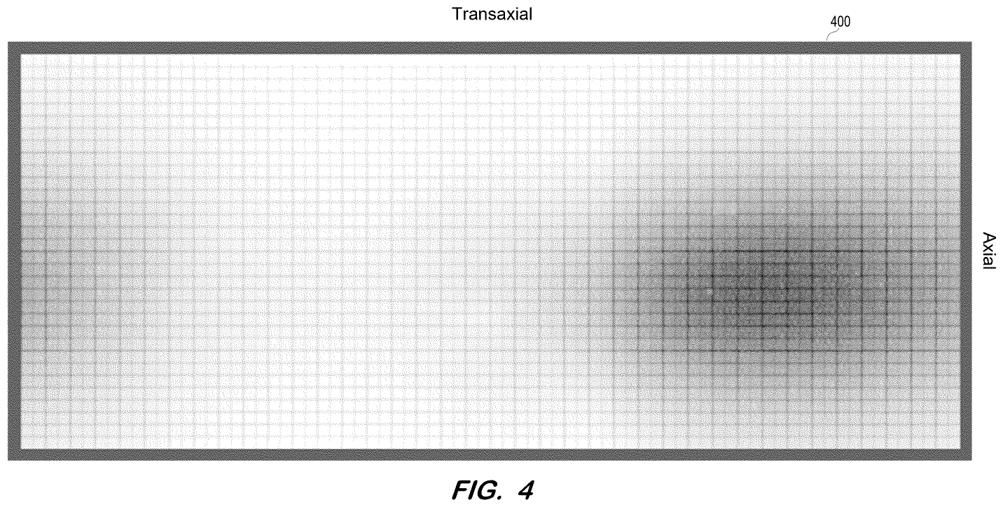

Singles count time-series data is determined for each of a plurality of PET crystals at S320 based on the PET singles data. FIG. 4 presents detector ring 400 in an unrolled configuration. Ring 400 includes 243,200 detector crystals, with rows of 320 detector crystals in the axial direction and rows of 760 detector crystals in the transaxial direction. Embodiments are not limited to the specific structure of detector ring 400. S320 may include determination, for each crystal of detector 400, of a count of singles received during each of a sequence of consecutive time periods (e.g., every 100 ms).

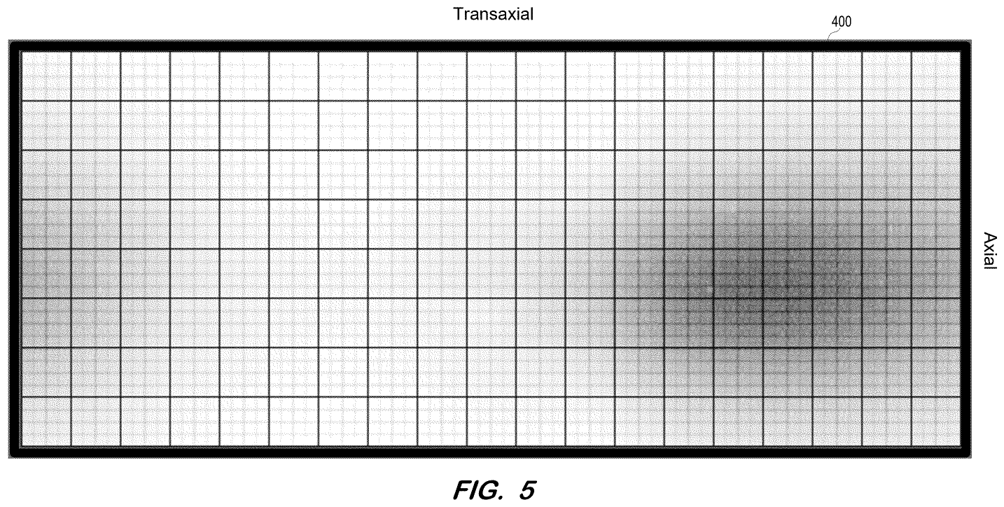

Singles count time-series data for each of a plurality of detector regions is determined at S330. FIG. 5 illustrates detector regions of detector ring 400 according to some embodiments. Each square region shown in FIG. 5 includes a respective 40×40 detector crystals. Singles count time-series data for a detector region is determined at S330 as a sum of the singles count time-series data for each detector crystal in the detector region. The detector regions for which singles count time-series data are determined at S330 may be a subset of less than all detector regions (and/or may include less than all detector crystals) of detector ring 400.

According to some embodiments, each detector region consists of a number (e.g., 8) of detector blocks. The singles count time-series data for each of the plurality of detector regions may therefore be determined at S330 by summing the singles count time-series data output by the scalers of the detector blocks of each detector region. This time-series data may represent a count of singles received every 250 ms in some embodiments.

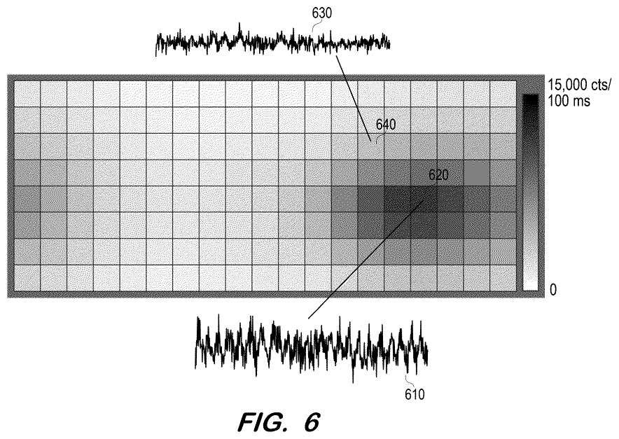

FIG. 6 illustrates singles count time-series data 610 for region 620 of detector crystals and total singles count time-series data 630 for region 640 of detector crystals according to some embodiments. Singles count time-series data 610 and 630 are plots of n(x, y, t), or the number of counts per 0.1 s at time t in region (x, y). Detector regions 620 and 640 are shaded to indicate an average number of counts per 100 ms represented by their corresponding time-series data 610 and 630.

S340 includes determination, from the singles count time-series data of each detector region, of the singles count time-series data which exhibits a largest variation. The variation of singles count time-series data of a detector region is a measure of an amount of patient motion represented by the singles count time-series data of the detector region. In some embodiments, S340 includes smoothing each time-series data according to any suitable smoothing algorithm, including but not limited to:

$${n}^{′} ( x , y , t ) = n ( x , y , t ) * {k}_{500 ms} ( t ) - n ( x , y , t ) * {k}_{10 s} ( t ) ,$$

where k₅₀₀ₘₛ and k_{10 s} are convolutional boxcar filtering kernels with half-widths of 500 ms and 10 s, respectively. The 10 s kernel width averages the waveform over a 20 s interval which is assumed to include several breathing cycles in most patients. n′ is an approximately zero-mean time series smoothed to remove variations on the order of 0.5 s.

A variation may be determined for each n′(x, y, t) as follows:

$${\sum }_{t = {t}_{1}}^{{t}_{2}} {{n}^{′} ( x , y , t )}^{2} .$$

In some embodiments, all time bins t of the time-series are used in the determination except for time bins of the first and last ten seconds of the time-series. A region (xₘₐₓ, yₘₐₓ) may be identified at S340 based on all the determined variations as:

$$( {x}_{max} , {y}_{max} ) = arg max {\sum }_{t = {t}_{1}}^{{t}_{2}} {{n}^{′} ( x , y , t )}^{2} ,$$

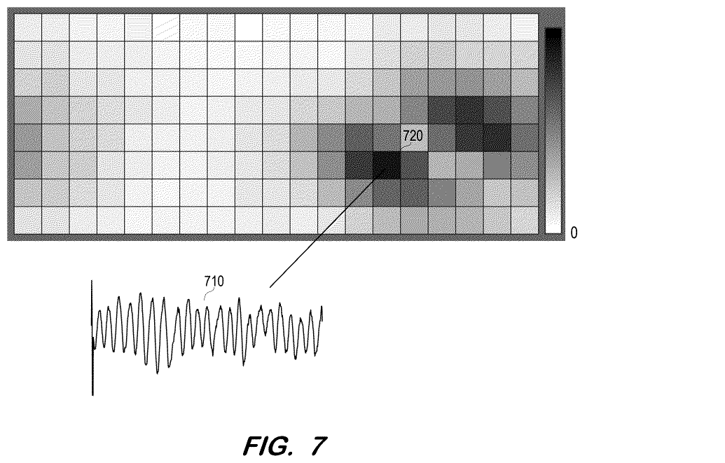

FIG. 7 illustrates smoothed singles count time-series data 710 of detector region 720 according to some embodiments. The shading of each detector region of FIG. 7 indicates the determined variation of its corresponding smoothed singles count time-series data. It will be assumed that singles count time-series data 710 of detector region 720 is determined to exhibit the largest variation at S340.

Next, at S350, correlations are determined between the singles count time-series data of each detector region and the singles count time-series data which exhibits the largest variation. According to the present example, correlations are determined at S350 between the singles count time-series data of each detector region and singles count time-series data 710 of detector region 720. Using the above notation, a correlation between region (xₘₐₓ>yₘₐₓ) and each region (x, y) may be described as correlation {n′ (x, y, t), n′ (xₘₐₓ) yₘₐₓ, t)}. In some embodiments of S350, and to increase processing speed, correlations are not determined for some detector regions (e.g., regions with low singles rates, regions outside the field of view, etc.).

The determined correlations may comprise values of any statistical measure of the relationship between two variables. In some embodiments, the determined correlations are in the range of −1 to +1. The determined correlations between time-series data X={x₁, x₂, . . . , x₁} and Y={y₁, y₂, . . . , yₙ} may comprise Pearson correlations calculated as follows:

$$r = \frac{\sum ( {x}_{i} - \overline{x} ) ( {y}_{i} - \overline{y} )}{\sqrt{\sum {( {x}_{i} - \overline{x} )}^{2} \sum {( {y}_{i} - \overline{y} )}^{2}}}$$

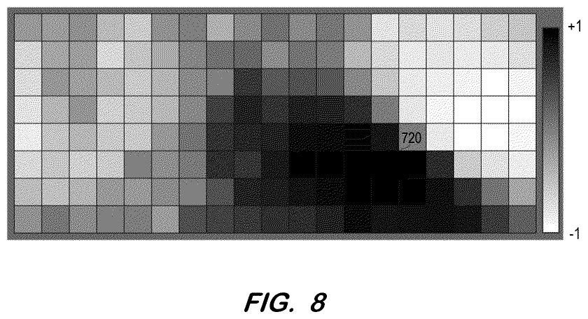

FIG. 8 illustrates values of correlations determined for each detector region at S350. A correlation value of +1 is determined for detector region 720. The darker-shaded detector regions may be considered in-phase with detector region 720 (i.e., their singles rate is positively correlated with the singles rate of detector region 720) while the lighter-shaded detector regions may be considered out-of-phase with detector region 720 (i.e., their singles rate is negatively correlated with the singles rate of detector region 720).

The singles count time-series data of each detector region is weighted based on its determined correlation at S360. In some embodiments, the singles count time-series data of each detector region is multiplied by its determined Pearson correlation at S360. In some embodiments, the singles count time-series data is subjected to a second smoothing algorithm prior to the weighting at S360, such as:

$${n}^{″} ( x , y , t ) = n ( x , y , t ) - n ( x , y , t ) * {k}_{10 s} ( t ) ,$$

with the 10 s kernel again selected to average the time-series over a 20 s interval. n″ is again an approximately zero-mean time-series, and retains more high temporal frequencies than n′. The weighted singles count time-series data for region (x, y) may then be represented as:

$${n}^{″} ( x , y , t ) \times correlation \{ {n}^{′} ( x , y , t ) , {n}^{′} ( {x}_{max} , {y}_{max} , t ) \} .$$

Any other suitable correlation-based weighting may be employed at S360.

A signal representing respiratory motion is generated at S370 based on the correlation-weighted time-series data of each detector region. S370 may comprise summing the weighted time-series data, i.e.,

$$w ( t ) = {\sum }_{x y} {n}^{″} ( x , y , t ) \times correlation \{ {n}^{′} ( x , y , t ) , {n}^{′} ( {x}_{max} , {y}_{max} , t ) \} ,$$

but embodiments are not limited thereto.

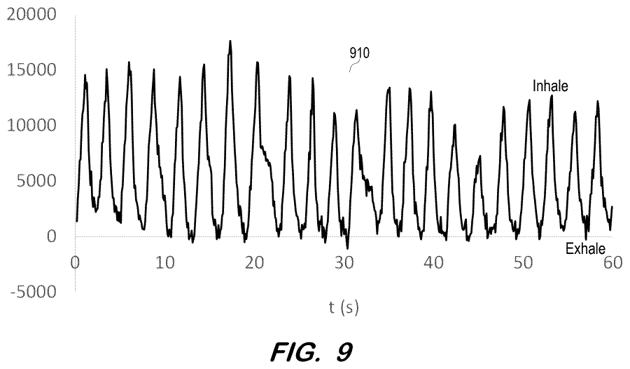

In some embodiments, the summed signal w (t) is inverted so that the signal peaks represent periods of inhalation and the signal valleys represent periods of exhalation. A baseline adjustment may also be applied such that all (or most) values of the summed signal are positive values. FIG. 9 illustrates signal 910 representing respiratory motion and generated based on correlation-weighted singles rate time-series data of each detector region according to some embodiments.

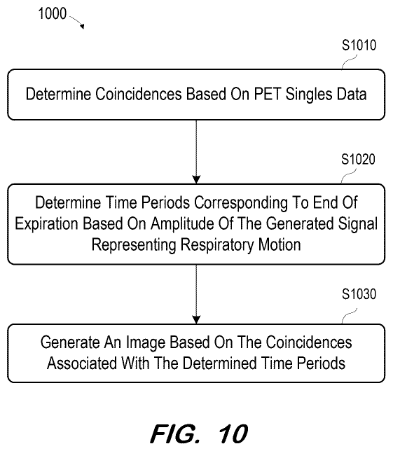

FIG. 10 is a flow diagram of process 1000 to generate gated PET images according to some embodiments. At S1010, coincidences are identified based on PET singles data. The PET singles data may represent the same singles detected at S310 of process 300 but, as described above, coincidences are identified at S1010 only from singles within a qualifying energy range (e.g., 425 keV-650 keV). The identified coincidences are timestamped with the time t at which they occurred.

At S1020, time periods corresponding to end of expiration portions of the respiratory motion are determined based on the amplitude of a signal representing respiratory motion, such as the signal generated at S370. For example, periods during which the amplitude of the signal is less than a threshold are determined and defined by their start time t₁ and their end time t₂.

Next, at S1030, an image is generated for based on the coincidences associated with the determined time periods. S1030 may comprise determining coincidences having timestamps falling within any (t₁, t₂) pair and applying any suitable reconstruction algorithm to the coincidences to generate an image therefrom. S1030 may also comprise incorporating additional coincidences into the generated image by warping and summing images generated based on other time periods (i.e., phases) as described above.

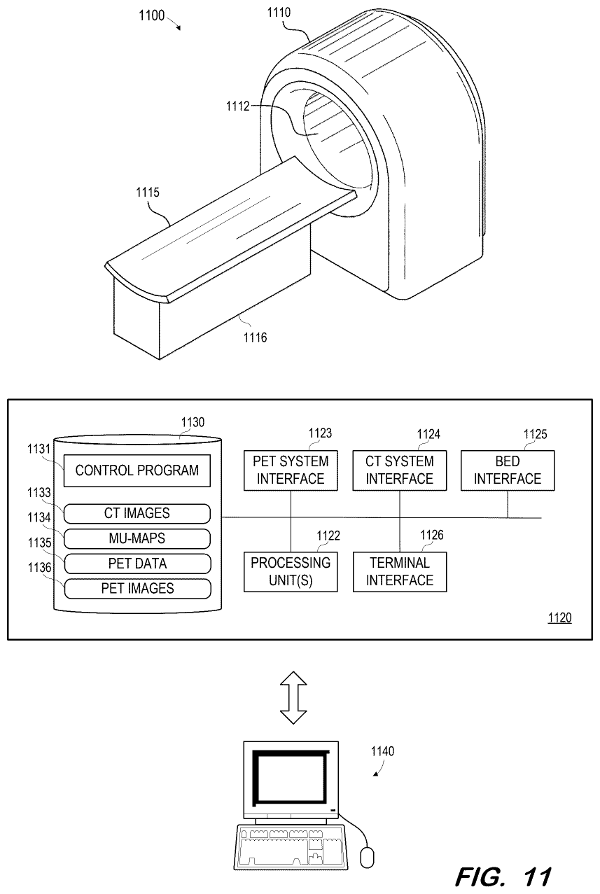

FIG. 11 illustrates PET-CT scanner 1100 to execute one or more of the processes described herein. Embodiments are not limited to scanner 1100 or to a multi-modality imaging system.

Scanner 1100 includes gantry 1110 defining bore 1112. As is known in the art, gantry 1110 houses PET imaging components for acquiring PET image data and CT imaging components for acquiring CT image data. The CT imaging components may include one or more x-ray tubes and one or more corresponding x-ray detectors as is known in the art. The PET imaging components may include any number or type of detectors and detector crystals disposed in any configuration as is known in the art.

Bed 1115 and base 1116 are operable to move a patient lying on bed 1115 into and out of bore 1112 before, during and after imaging. In some embodiments, bed 1115 is configured to translate over base 1116 and, in other embodiments, base 1116 is movable along with or alternatively from bed 1115.

Movement of a patient into and out of bore 1112 may allow scanning of the patient using the CT imaging elements and the PET imaging elements of gantry 1110. Bed 1115 and base 1116 may provide continuous bed motion and/or step-and-shoot motion during such scanning according to some embodiments.

Control system 1120 may comprise any general-purpose or dedicated computing system. Accordingly, control system 1120 includes one or more processing units 1122 configured to execute program code to cause system 1120 to acquire image data and generate images therefrom, and storage device 1130 for storing the program code. Storage device 1130 may comprise one or more fixed disks, solid-state random-access memory, and/or removable media (e.g., a thumb drive) mounted in a corresponding interface (e.g., a Universal Serial Bus port).

Storage device 1130 stores program code of control program 1131. One or more processing units 1122 may execute control program 1131 to control CT imaging elements of scanner 1100 using CT system interface 1124 and bed interface 1125 to acquire CT data and to reconstruct CT images 1133 therefrom. One or more processing units 1122 may execute control program 1131 to, in conjunction with PET system interface 1123 and bed interface 1125, control hardware elements to inject a radiopharmaceutical into a patient, move the patient into bore 1112 past PET detectors of gantry 1110, and acquire PET data 1134 as described above.

Control program 1131 may also be executed to determine a respiratory waveform signal based on PET singles data as described above. The respiratory waveform signal may be used to associate detected coincidences of PET data 1134 with different periods of respiratory motion.

Mu-maps 1135 may be derived from CT images 1133 and used to reconstruct PET images 1136 from PET data 1134. PET images 1136 and CT images 1133 may be transmitted to terminal 1140 via terminal interface 1126. Terminal 1140 may comprise a display device and an input device coupled to system 1120. Terminal 1140 may display the received PET images 1136 and CT images 1134. Terminal 1140 may receive operator input for selecting a region of interest, controlling display of the data, operation of scanner 1100, and/or the processing described herein. In some embodiments, terminal 1140 is a separate computing device such as, but not limited to, a desktop computer, a laptop computer, a tablet computer, and a smartphone.

Each component of scanner 1100 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

What is claimed is:

1. A system comprising:
a plurality of positron emission tomography (PET) detectors, each of the PET detectors comprising a plurality of detector crystals,
the system to:
detect singles at each of a plurality of regions of detector crystals;
for each of the plurality of regions, determine time-series data of a count of singles detected at the region;
determine a motion-representative region based on the time-series data of the count of singles determined for each region;
determine a correlation between the time-series data of the count of singles determined for the motion-representative region and time-series data of the count of singles determined for each of one or more other regions of the plurality of regions;
generate a motion signal based on the time-series data of the count of singles determined for the motion-representative region and the time-series data of the count of singles determined for each of the one or more other regions of the plurality of regions based on the determined correlations;
determine coincidences corresponding to selected periods of the motion signal; and
reconstruct an image based on the determined coincidences.

2. The system of claim 1, wherein generation of the motion signal comprises combination of the time-series data of the count of singles determined for the motion-representative region and the time-series data of the count of singles determined for each of the one or more other regions based on the determined correlations.

3. The system of claim 2, wherein combination of the time-series data of the count of singles determined for the motion-representative region and the time-series data of the count of singles determined for each of the one or more other regions comprises:
summing an inverse of the time-series data of the count of singles determined for each of a first plurality of the one or more other regions with the time-series data of the count of singles determined for each of a second plurality of the one or more other regions.

4. The system of claim 3, wherein combination of the time-series data of the count of singles determined for the motion-representative region and the time-series data of the count of singles determined for each of the one or more other regions based on the determined correlations comprises smoothing the time-series data of the count of singles determined for each of the plurality of regions and combining the smoothed time-series data based on the determined correlations.

5. The system of claim 1, wherein determination of the motion-representative region comprises smoothing the time-series data of the count of singles determined for each of the plurality of regions and determining a sum of each of the smoothed time-series data.

6. The system of claim 5, wherein determination of the motion-representative region comprises determination that the sum of the smoothed time-series data of the count of singles determined for the motion-representative region is of larger magnitude than the sum of the smoothed time-series data of the count of singles determined for each of the one or more other regions.

7. The system of claim 1, wherein the detected singles exhibit an energy range between 150-250 keV and were emitted from a yttrium-90 tracer.

8. The system of claim 1, wherein determination of the correlation comprises determination of a Pearson correlation between the time-series data of the count of singles determined for the motion-representative region and time-series data of the count of singles determined for each of the one or more other regions.

9. The system of claim 8, wherein generation of the motion signal comprises combination of the time-series data of the count of singles determined for the motion-representative region and the time-series data of the count of singles determined for each of the one or more other regions by weighting the time-series data of the count of singles determined for each of the one or more other regions by the Pearson correlation between the time-series data of the count of singles determined for each of the one or more other regions and the time-series data of the count of singles determined for the motion-representative region.

10. A method comprising:
acquiring data representing singles received by a plurality of regions of detector crystals;
for each of the plurality of regions, determining time-series data of a count of singles detected by the detector crystals of the region;
determining a representative region based on the time-series data of the count of singles detected by the detector crystals of each region;
determining a correlation between the time-series data of the count of singles determined for the representative region and time-series data of the count of singles determined for each other one of the plurality of regions;
generating a motion signal based on the time-series data of the count of singles determined for the representative region and the time-series data of the count of singles determined for each other one of the plurality of regions based on the determined correlations;
determining coincidences corresponding to selected periods of the motion signal; and
reconstructing an image based on the determined coincidences.

11. The method of claim 10, wherein generating the motion signal comprises combining the time-series data of the count of singles determined for the representative region and the time-series data of the count of singles determined for each other one of the plurality of regions based on the determined correlations.

12. The method of claim 11, wherein combining the time-series data of the count of singles determined for the representative region and the time-series data of the count of singles determined for each other one of the plurality of regions comprises:
summing an inverse of the time-series data of the count of singles determined for each of a first plurality of the one or more other regions with the time-series data of the count of singles determined for each of a second plurality of the one or more other regions.

13. The method of claim 12, wherein combining the time-series data of the count of singles determined for the representative region and the time-series data of the count of singles determined for each other one of the plurality of regions based on the determined correlations comprises smoothing the time-series data of the count of singles determined for each of the plurality of regions and combining the smoothed time-series data based on the determined correlations.

14. The method of claim 10, wherein determining the representative region comprises smoothing the time-series data of the count of singles detected by the detector crystals of each of the plurality of regions and determining a sum of the smoothed time-series data for each of the plurality of regions.

15. The method of claim 14, wherein determining the representative region comprises determining that the sum of the smoothed time-series data of the count of singles determined for the representative region is of larger magnitude than the sum of the smoothed time-series data of the count of singles determined for each other of the plurality of regions.

16. The method of claim 10, wherein the detected singles exhibit an energy range between 150-250 keV and were emitted from a yttrium-90 tracer.

17. The method of claim 10, wherein determining the correlation comprises determining a Pearson correlation between the time-series data of the count of singles determined for the representative region and time-series data of the count of singles determined for each other of the plurality of regions.

18. The method of claim 17, wherein generating the motion signal comprises combining the time-series data of the count of singles determined for the representative region and the time-series data of the count of singles determined for each other of the plurality of regions by weighting the time-series data of the count of singles determined for each of the plurality of regions by the Pearson correlation between the time-series data of the count of singles determined for each of the plurality of regions and the time-series data of the count of singles determined for the representative region.

19. One or more computer-readable media storing program code executable by one or more processing units of a computing system to cause the computing system to perform operations comprising:
for each of a plurality of PET detector regions, determining time-series data of a count of singles detected at the region;
determining a representative region based on the time-series data of the count of singles determined for each region;
determining a correlation between the time-series data of the count of singles determined for the representative region and time-series data of the count of singles determined for each other of the plurality of regions;
generating a motion signal based on the time-series data of the count of singles determined for the representative region and the time-series data of the count of singles determined for each other of the plurality of regions based on the determined correlations;
determining coincidences corresponding to selected periods of the motion signal; and
reconstructing an image based on the determined coincidences.

20. The one or more computer-readable media of claim 19, wherein combining the time-series data of the count of singles determined for the representative region and the time-series data of the count of singles determined for each other of the plurality of regions comprises:
summing an inverse of the time-series data of the count of singles determined for each of a first plurality of the one or more other regions with the time-series data of the count of singles determined for each of a second plurality of the one or more other regions.
